(19) United States
(12) Patent Application Publication
(10) Pub. No.: US 2007/0003579 A1
(43) Pub. Date: Jan. 4, 2007

(54) Allergen-specific immunotherapy

(75) Inventor: Thomas M. Kundig, Bassersdorf (CH)

Correspondence Address: BANNER & WITCOFF, 1001 G STREET N W, SUITE 1100, WASHINGTON, DC, 20001, US

(73) Assignee: MannKind Corporation, Valencia, CA (US)

(21) Appl. No.: 11/170,063

(22) Filed: Jun. 30, 2005

(51) Int. Cl. A61K 39/35; A61K 39/36

(52) U.S. Cl. 424/275.1

(57) ABSTRACT

Heat-denatured allergen compositions of bee venom, grass pollen, birch pollen, and cat dander or hair for use in allergen-specific immunotherapy.

### FIELD OF THE INVENTION

The invention relates to allergen-specific immunotherapy.

### BACKGROUND OF THE INVENTION

Type-I hypersensitivity is mediated through IgE bound to the surfaces of mast cells and basophils. Subsequent exposure to the allergen can cross-link the IgE and cause degranulation of the cells with the release of histamines and other mediators that instigate allergic symptoms. Current treatment is generally symptomatic, and the only causative treatment that has a long-lasting effect is allergen-specific immunotherapy (SIT), which typically involves numerous (30-50) subcutaneous injections with the respective allergen extract over a time period of 3-5 years. Such conventional SIT is therefore time consuming, costly, and bears a high risk of allergic side effects.

Currently, recombinant allergens have received much attention due to the fact that functional groups that mediate toxic or allergic side effects can be deleted. This furthermore allows the use of higher therapeutic doses, which per se is known to promote Th1 immune responses (Boonstra et al., J Exp Med 197:101., 2003; Von Gamier et al., Clin Exp Allergy 32:401, 2002) and to be favorable in fighting allergic immunopathology (Andre et al., Int Arch Allergy Immunol 131:111, 2003; Ewbank et al., J Allergy Clin Immunol 111:155, 2003). However, recombinant technology is costly and inefficient as compared to the production of allergen extracts from, for example, grass or tree pollens. Furthermore, the application of recombinant allergens might be limited to species with only one or very few protein allergens, e.g., cat dander. Most species contain a multiple of potential protein allergens, e.g., pollen, for which production and testing of the corresponding recombinant allergens will generate high-cost vaccines.

There is a continuing need in the art to for safe and effective means of desensitizing individuals to allergens.

### BRIEF DESCRIPTION OF THE FIGURES

FIG. 1. Mice were immunized four times every two weeks with 0.2 μg native or heat-denatured BV in Al(OH)₃ by intralymphatic injections. Anti-PLA2 IgG1, IgG2a and IgE antibodies were measured by ELISA. Representative results from three experiments (n=3-4 mice) show titration curves of sera prepared 14 weeks after the last and first dose, respectively.

FIG. 2. Mice (n=3) were sensitized with 0.3 μg native (filled symbols and bars) or heat-denatured (open symbols and bars) BV (A, C) or ovalbumin (B, D) in Al(OH)3 by seven weekly intraperitoneal injections. Anti-PLA2 antibody isotypes were measured by ELISA. Representative results from two experiments show titration curves of sera prepared 10 weeks after the priming dose. Twelve weeks after priming and six weeks after the last injection, mice were challenged with 30 μg PLA2 (C) or 300 μg ovalbumin (D) intraperitoneally and anaphylaxis was measured as temperature drop after 30 minutes.

FIG. 3. Mice were immunized four times every two weeks by intralymphatic injections of native (filled symbols) or heat-denatured (2h 80° C.; open symbols) cat dander extract (A), grass pollen allergen extract (B) or birch pollen allergen extract (C) in Al(OH)33. IgG1 and IgG2a antibodies were measured by ELISA 12 weeks after the priming dose using the allergen extract as coating allergen. Comparable results were obtained at earlier time points (not shown).

FIG. 4. Mice were immunized with 0.3 μg native or differently heat-denatured BV in Al(OH)3 four times every two weeks by intralymphatic injections (filled symbols). Alternatively, the same vaccine modifications were added another 0.03 μg native BV prior to injection. Anti-PLA2 IgG1 and IgG2a antibodies were measured by ELISA 10 weeks after the priming dose.

FIG. 5. Mice were immunized as described in FIG. 4 with 0.3 μg native or differently heat-denatured BV in Al(OH)33 (filled bars) or the same vaccines with the addition of 0.03 μg native BV prior to injection (open bars). Anti-PLA2 IgG1 and IgG2a antibodies were measured by ELISA 10 weeks after the priming dose.

FIG. 6. In vitro inhibition of human IgG4 (A,B) and IgE (C,D) by native (filled symbols) and heat-denatured (open symbols) PLA2 (A,C) and bee venom (B,D). The results are representative of three experiments.

### DETAILED DESCRIPTION OF THE INVENTION

Heat-denatured allergen compositions of the invention stimulate strong Th1-type immune responses characterized by IgG2a isotype production. This stimulation correlates with weakened allergy in a therapeutic model. While not wishing to be bound by these explanations, it appears that heat denaturation damages crucial IgE binding epitopes and simultaneously promotes the exposure of T-cell epitopes which facilitate stimulation of T-helper type 1 (Th1) CD4+ T cells. Another possible explanation may be that coagulation due to heat denaturation turns the allergens into a particulate form that enhances phagocytosis and processing by professional antigen presenting cells. Despite their reduced IgE binding, denatured allergens are still recognized by human IgG4 equally well as native allergens. As induction of IgG4 is discussed as the mechanism by which SIT protects the allergic individual against the allergen, this demonstrates the potential of heat-denatured allergens in SIT.

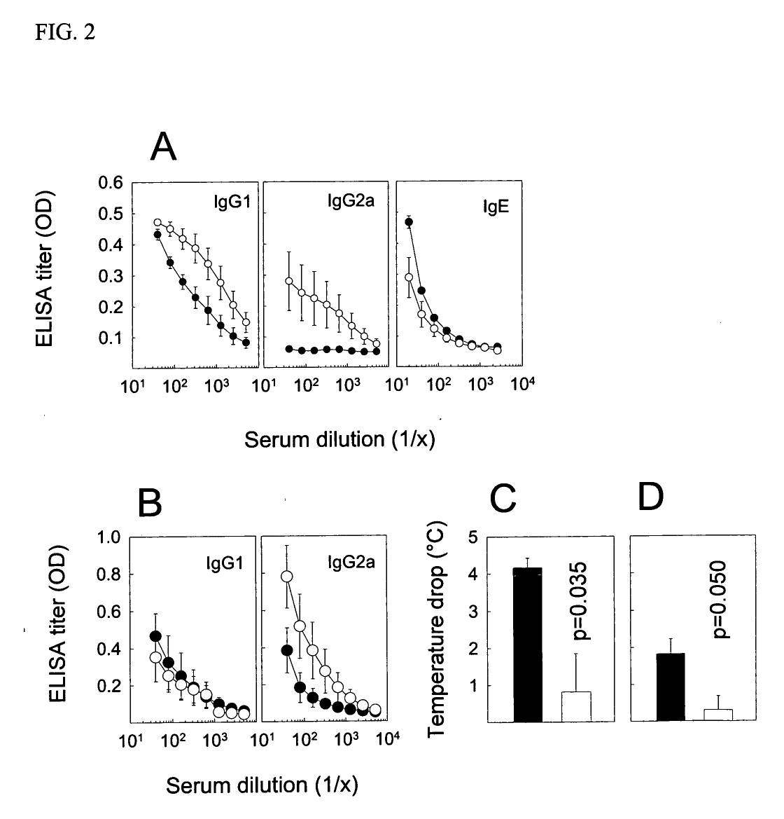

Both IgE production and IgE-binding capacity can be reduced upon heat-denaturation of allergen extracts or purified proteins. This changed property is of utmost importance for the immunotherapeutic safety of the allergy vaccine. The superior safety of heat-denatured allergens is further underlined by its lower sensitization potential in mice. Animals immunized intraperitoneally with heat-denatured bee venom allergen extract or ovalbumin not only produce less IgE than their littermates which received the native allergen, but they also suffered less anaphylaxis upon a challenge of the native allergen. FIG. 2.

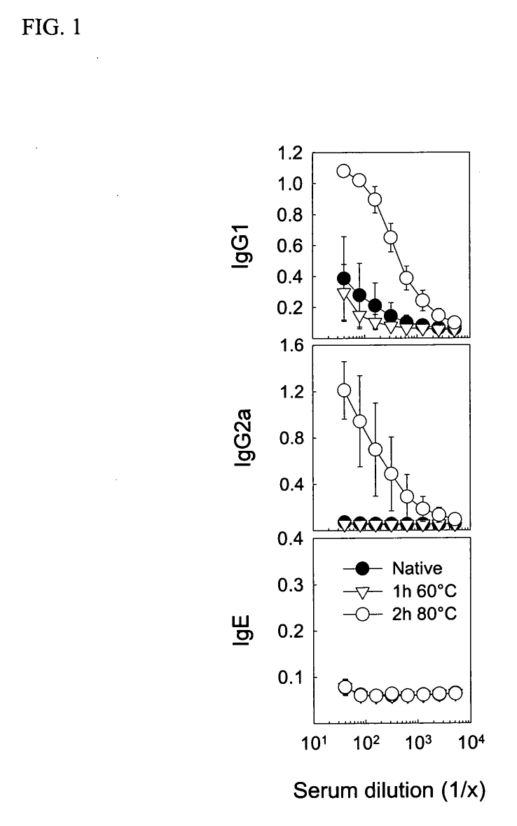

A vaccine used for SIT not only must be safe, it must provide means for de-sensitization, i.e., the heat-denatured allergens should also be able to produce IgGs (especially, IgG2a in mice) and a cytokine environment favorable for the protection against a subsequent natural exposure to allergens (1). Rather strikingly, as demonstrated in the specific examples described below, only the heat-denatured allergens produced IgG2a (FIGS. 1-3). In contrast, mice immunized with native bee venom extract, bee venom PLA2, birch pollen extract, grass pollen extract, or cat dander extract raised high titers of IgG1 but not IgG2a.

Heat-denatured allergen compositions represent a novel and potential medical and socio-economical alternative in allergen-specific immunotherapy. Heat-denatured allergen compositions comprising allergen extracts are particularly cost-effective. The extracts are easily available and produced, the denaturation is simple and of practically no cost, and the product is highly safe and immunogenic with diminished IgE binding capacity. The product also skews the immune response towards Th1, which leads to production of IgG2a antibodies that protected mice from anaphylaxis in a therapeutic model. Moreover, the potency of heat-denatured allergens permits reducing the therapeutic allergen dose by orders of magnitude, which again is of uttermost importance for the SIT safety.

Heat-Denatured Allergen Compositions

Heat-denatured allergen compositions of the invention preferably comprise one or more allergens found in bee venom (e.g., phospholipase A2 (Api m 1), hyaluronidase (Api m 2), Api m 6), in cat dander or hair (e.g., Fel d 1; cystatin), in grass pollen (e.g., Phl p 1, 2, 4, 5, 6, 7, 11, 12), or in birch pollen (e.g., Bet v 1 and Bet v 2) and a physiologically acceptable vehicle.

In some embodiments the allergen is present in an extract of bee venom, cat dander or cat hair, grass pollen, or birch pollen. An “extract” is a preparation which contains components other than the allergen and can be prepared by methods known in the art. For example, grass pollen allergen extracts can be prepared as described in Rossi & Monasterolo, Int Arch Allergy Immunol. 2004 September; 135(1):44-53. Cat dander or hair extracts can be prepared as described in Nanda et al., J Allergy Clin Immunol. 2004 December; 114(6):1339-44; Genin et al., Allergy. 1994 September; 49(8):645-52; Wahl & Maasch, Engl Reg Allergy Proc. 1987 July-August; 8(4):237-43; Hjelmroos et al., Allerg Immunol (Paris). 1993 April; 25(4):137-40. Bee venom extracts include extracts of whole bees, venom sacs, and whole bee venom. Bee venom extracts can be prepared as described, for example, in Lima et al., J. Venom. Anim. Toxins, 2000, vol. 6, no. 1, p. 64-76. Birch pollen extracts can be prepared as described in Cadot et al., Allergy. 1995 May; 50(5):431-7; Vik et al., Ann Allergy. 1987 January; 58(1):71-7. Allergen extracts also can be obtained commercially.

In other embodiments the allergen is a purified allergen. “Purified” in this context means at least partially separated from components associated with the allergen in its natural source, such as other proteins, lipids, nucleic acids, subcellular components, etc. Allergens can be purified from a natural source by any suitable means including, but not limited to, size exclusion chromatography, ammonium sulfate fractionation, ion exchange chromatography, affinity chromatography, and preparative gel electrophoresis. A preparation of purified allergen is at least 80% pure (e.g., at least 80% of the material in the preparation is the allergen); preferably, the preparations are 90%, 95%, or 99% pure. Purity of the preparations can be assessed by any means known in the art, such as SDS-polyacrylamide gel electrophoresis.

Purification of bee venom allergens is described, for example, in Kettner et al., J Allergy Clin Immunol. 2001 May; 107(5):914-20 (Api m 6), and Kettner et al., Clin Exp Allergy. 1999 March; 29(3):394-401 (PLA2). Purification of grass pollen allergens is described in Corti et al., Proteomics. 2005 February; 5(3):729-36. Purification of birch pollen allergens is described in Vik et al., Ann Allergy. 1987 January; 58(1):71-7; Hjelmroos et al., Allerg Immunol (Paris). 1993 April; 25(4):137-40. Purification of cat allergen Fel d 1 is described in van Ree et al., J Allergy Clin Immunol. 1999 December; 104(6):1223-30.

Alternatively, a purified allergen can be produced recombinantly or can be synthesized chemically using well-known methods. Chemical synthesis methods are well known in the art. See, e.g., Merrifield, J. Am. Chem. Soc. 85, 2149-2154, 1963; Roberge et al., Science 269, 202-204, 1995; and WO 01/98340.

Recombinant production of group I grass pollen allergens is described, for example, in Ball et al., FEBS J. 2005 January; 272(1):217-27, and Rossi & Monasterolo, Int Arch Allergy Immunol. 2004 September; 135(1):44-53. Recombinant production of bee venom allergens is described, for example, in Markovic-Housley et al., Structure Fold Des. 2000 Oct. 15; 8(10):1025-35 (hyaluronidase) and Muller et al., Clin Exp Allergy. 1997 August; 27(8):915-20 (PLA2). Recombinant production of birch and other tree pollen allergens is described in Erdmann et al., Int Arch Allergy Immunol. 2005 March; 136(3):230-8 and Jahn-Schmid et al., Clin Exp Allergy. 2003 October; 33(10):1443-9. Recombinant production of the cat allergen Fel d 1 is described in Seppala et al., J Biol Chem. 2005 Feb. 4; 280(5):3208-16, and Kaiser et al., Acta Crystallogr D Biol Crystallogr. 2003 Jun.; 59(Pt 6):1103-5. A purified allergen also can be a recombinantly produced fusion protein. See, e.g., Kussebi et al., J Allergy Clin Immunol. 2005 February; 115(2):323-9.

Heat-denatured allergen compositions can comprise one or more allergens (either purified allergens or allergen extracts) from each of bee venom, cat dander or cat hair, grass pollen, or birch pollen or can comprise a mixture of such allergens (e.g., a grass pollen and a birch pollen allergen). The concentration of a purified allergen typically varies from 0.1-20 microgram per milliliter. The concentration of an allergen extract in such compositions typically varies from 1-200 microgram per milliliter.

The physiologically acceptable vehicle can be any vehicle typically used with vaccines or allergy shots, such as water, standard saline solutions, dextrose solutions, and water comprising albumin. The physiologically acceptable vehicle preferably is pyrogen-free.

Adjuvants

Optionally, a heat-denatured allergen composition can be delivered in combination with an adjuvant. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; immunomodulators such as cytokines, interleukins (e.g., IL-1, IL-2, IL-4, IL-5, IL-6, IL-7, IL-12, etc.), interferons (e.g., interferon-γ), macrophage colony stimulating factor, and tumor necrosis factor; alum; BCG; mineral-containing compositions (e.g., mineral salts, such as aluminium salts and calcium salts, hydroxides, phosphates, sulfates, etc.); bioadhesives and mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol). See WO 02/34771. One or more of these components can be added to enhance the response to the allergen, increase adsorption of the heat-denatured allergen, provide increased comfort to the patient, and/or slow the release of the heat-denatured allergen to prolong exposure.

Methods of Preparing Heat-Denatured Allergen Compositions

Heat-denatured allergen compositions of the invention typically are prepared by heating an allergen preparation comprising, e.g., a bee venom, grass pollen, birch pollen, or cat dander or hair allergen, for a time sufficient to (a) reduce allergen's ability to bind IgE, (b) reduce the allergen's ability to produce IgE, and (c) increase the allergen's ability to produce IgG. The allergen's ability to bind IgE preferably is reduced by more than 90 percent, or between 50 and 99.9%. The allergen's ability to increase production of IgE preferably is reduced by more than 90 percent, or between 50 and 99.9%. The allergen's ability to increase production of an IgG preferably is increased by more than a factor of 10, or between 2 and 1000 fold. Methods of detecting these effects are well known in the art. See also the specific examples, below.

In some embodiments an allergen preparation is heated method at a temperature between 60 and 99° C., between 40 and 99° C., or between 40 and 121° C., or between 40 and 180° C. (e.g., at about 120° C. for 20 minutes, about 121° C. for 15 minutes, at about 160° C. for 60 minutes, or about 180° C. for 20 minutes). The heating time and temperature for any particular allergen preparation is readily determined by the skilled practitioner. See the specific examples, below.

Methods of Desensitizing an Individual to an Allergen

Heat-denatured allergen compositions of the invention can be used to desensitize individuals, particularly humans, to allergens contained within the compositions. An effective amount of such a composition is an amount which (a) reduces an allergen's ability to bind IgE, (b) reduce the allergen's ability to increase production of IgE, and (c) increase the allergen's ability to increase production of an IgG. Preferably, an effective amount of the composition reduces one or more of the patient's symptoms, such as swelling, itching, redness, watery eyes, sneezing, wheezing, nasal congestion, etc. A patient can be tested for baseline reactions before administration begins, using assays such as those which measure IgG and IgE levels, T-cell stimulation, basophil degranulation, and/or controlled allergen exposures, such as skin tests, nasal and conjunctival provocation tests, and bee- or wasp sting challenges. To determine whether a patient has been desensitized, one or more of these measurements can be compared to measurements taken after administration. Examples of suitable assays are provided in U.S. Pat. No. 6,773,695. It is well within the skill of the art to identify and employ alternative assays.

Modes of Administration

Heat-denatured allergen compositions for desensitization treatment and/or maintenance treatment can be administered by any suitable means, including subcutaneous injection, sublingual application, and intranodal injection. In preferred embodiments of the invention, the heat-denatured allergen composition is delivered directly to a lymph node during both desensitization treatment and maintenance treatment. See U.S. Pat. No. 6,773,695. Alternatively, the heat-denatured allergen composition can be delivered directly to the lymph node during the desensitization phase and subcutaneously during the maintenance phase. Although less preferred, some of the benefits of the invention can be conferred if intranodal therapy is employed during desensitization and subcutaneous therapy is employed during maintenance.

Doses

A heat-denatured allergen composition can be delivered in a dose comprising about 0.001 μg to 1000 μg and more preferably in a dose from about 0.01 μg to about 100 μg, although the optimal dose will vary depending on the allergen being injected, the weight of the patient, the immune system of the patient, and the like. Effective treatment in many cases can be accomplished with one delivery. In some embodiments, treatment includes from 1 to 15 injections. In preferred embodiments, treatment includes from 1 to 5 injections and more preferably 1 to 3 injections. Treatment can also be maintained by one annual injection of the desensitization preparation. For example, the standard escalation after a test dose of 0.1 μg involves administration of 1 μg followed by 5 μg and 10 μg. Escalation depends on the patient's tolerance of the previous dose. Multiple injections can be delivered periodically, e.g., over a course of days, once or twice per month, or several times per year. Also, so called rush or ultra-rush regimens may be used, where several doses are injected within one day (rush) or within a few hours (ultra-rush), typically within 2-4 hours.

The dose employed during the initial (desensitization) phase can be from 0.01 μg to 1 μg delivered in from 1 to 5, preferably from 1 to 3, injections of 1 μg, 5 μg and 10 μg over the course of from several days up to 3 months. In preferred embodiments, the allergen is delivered 2 to 3 times, 1 to 2 weeks apart. During desensitization treatment, 50 μl to 200 μl of a heat-denatured allergen composition is administered directly into the lymph node starting with very small doses of allergen, from 0.01 μg up to 10 μg. This dose is one-tenth the normal dose for subcutaneous immunotherapy, and therefore the possibility of side effects is minimized.

The dose employed during the maintenance phase can be from 0.01 μg to 50 μg, preferably 0.01 μg to 20 μg, delivered periodically over the course of from several months to several years. During maintenance treatment, for example, a patient's lymph node is injected with from 0.01 μg to 50 μg of allergen in injections of typically 10 μl to 200 μl each.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

Immunization Protocols

Young female CBA/J mice from Harlan (Horst, The Netherlands) were immunized with native or heat-denatured extracts of bee venom and grass pollen (ALK-Abello) and cat dander (Stallergenes) all purchased from Trimedal Ltd (Bruttisellen, Switzerland). Ovalbumin was from Sigma Aldrich (Buchs, Switzerland). Denaturation of concentrated solutions of allergens in saline containing human serum albumin at 60, 80 and 99° C. under agitation was performed using a Compact Thermomixer from Eppendorf.

The injected allergens were injected with 30% (V/V) of a two percent suspension of aluminium hydroxide (ALU-GEL-S®, Serva, Heidelberg, Germany) in phosphate-buffered saline. For intraperitoneal and intralymphatic injections, 50 and 10 microliter, respectively, of the vaccine was injected; intralymphatic injections were performed on anesthetized (Ketamine and Xylazine) mice. Serum was prepared from clotted blood collected from the tail vein at different time points and frozen at −20° C. until analyzed by ELISA.

For induction of anaphylactic responses, immunized mice were injected intraperitoneally with 30 μg of the purified bee-venom major allergen phospholipase A2 (PLA2) from Sigma Aldrich or 300 μg ovalbumin. Rectal temperature was monitored with a calibrated digital thermometer after 30 minutes.

### EXAMPLE 2

Antibody Determination

For detection of antibodies, microtitre 96-well plates (Nunc Maxisorb) were coated at 4° C. overnight with 100 μl of 5 μg/ml of PLA2, 1 μg/ml whole birch pollen extract, 1 μg/ml whole cat dander allergen extract, or 2 μg/ml whole grass pollen extract, respectively, in buffered carbonate (pH 9.4). Plates were washed with PBS-0.05% Tween 20 (PBST) and saturated with 150 μl of 2.5% non-fat dry milk (PBSTM) at room temperature (RT) for one hour. After washing, serial dilutions of individual sera in 100 μl PBSTM were added to the plates, which were then incubated at RT for two hours. Plates were washed and then incubated with 1 μg/ml goat anti-mouse IgG1 or IgG2a conjugated to biotin (BD Biosciences Pharmingen, San Diego, Calif.) in 100 μl PBSTM at RT and for two hours. After further washing and incubation with 100 μl of a 1:1000 dilution of strepatavidin-conjugated HRP (SA-HRP; BD Pharmingen) at RT for one hour, the plates were washed again and added 100 μl of the enzyme substrate 2,2′-azino-bis(3-ethylbenzothiazoline-6-sulfonic acid) diammonium salt (ABTS; Sigma) in 1 M sodium dihydrogen phosphate. The absorption was read at 405 nm after 20 min incubation of the substrate.

For detection of PLA2-specfic IgE, plates were coated with 5 μg/ml of a mouse anti-IgE antibody. As secondary antibody to bound mouse serum, biotinylated PLA2 was used at 3 μg/ml.

### EXAMPLE 3

Inhibition Assay

To evaluate the recognition of native and heat-denatured allergens by human antibodies, the allergens were tested in an ELISA inhibition assay and in a Western blot assay. The inhibition assay was performed by coating 96-well plates at 4° C. overnight with 100 μl of a 5 μg/ml PLA2 solution in buffered carbonate, pH 9.4. The plates were then blocked with PBSTM. In a second series of plates, two-fold dilutions of the native and heat-denatured allergens were prepared at 60 μl. These were incubated at 37° C. and for 2 hours with 60 μl of 1:400 dilutions of a sero-positive human serum against bee venom. Subsequently, 100 μl of the antisera-allergen solutions were transferred to the washed and emptied PLA2-coated and blocked plates for further incubation at 37° C. for 2 hours. After discarding the solution and washing the plates, 100 μl of biotinlyated anti-human IgG4 or IgE antibodies were added for 1.5 hour as recommended by the provider (BD Pharmingen). The plates were finally developed with SA-HRP (1:1000) and ABTS as described above.

### EXAMPLE 4

Heat-Denaturation of Bee Venom Extract Strongly Enhances its Immunogenicity

The effect of heat-denaturation on the immunogenicity of an allergen was evaluated in CBA mice immunized four times every two weeks by intralymphatic injections of 0.2 μg native or denatured bee venom (BV) in saline. The denatured BV was prepared by either heating a 0.1 mg/ml aqueous solution on a Thermoblock at 60° C. or 80° C. for one or two hours, respectively. As illustrated in FIG. 1, native BV with aluminium hydroxide was only weakly immunogenic under the described experimental conditions and only IgG1, an isotype that is dependent on Th2, was induced. Heat-denaturation at 60° C. for one hour insignificantly improved IgG1 induction. However, more excessive denaturation at 80° C. for two hours had a highly significant effect on the immunogenicity. High titers of IgG1 were obtained and most importantly, very high titers of the Th1-dependent isotype IgG2a were induced. None of the preparations induced detectable IgE antibodies against PLA2. Blood samples drawn at time points earlier than the illustrated 14 weeks showed similar results.

The immunogenicity of heat-denatured bee venom allergen was further studied after intraperitoneal administration, which predominantly induces IgE. In contrast to intralymphatic injections (FIG. 1), intraperitoneal injections of 0.3 μg native BV stimulated strong IgG1 responses (FIG. 2A). Heat-denaturation of BV at 80° C. for two hours enhanced IgG1 production (p<0.05 for dilutions 1/80 through 1/640). Also, while native BV did not induce IgG2a, heat-denaturation again caused major IgG2a production, a phenomenon that is unexpected for low-dose intraperitoneal immunization. Finally, heat-denaturation also reduced the capability of BV to induce IgE in mice upon intraperitoneal administration (p<0.05 for dilutions 1/20 through 1/80). Blood samples drawn at earlier time points than the illustrated 10 weeks showed similar results.

To analyze whether the effect of heat-denaturation was a particular characteristic of BV, we treated CBA mice with seven weekly intraperitoneal injections of 0.3 μg ovalbumin. Although less significant than for BV, heat-denaturation (80° C., two hours) caused improved IgG2a production but no changes in the IgG1 production (FIG. 2B).

A common method for testing allergy in mice is to induce anaphylaxis by means of high doses of systemically administered allergens. We therefore injected the mice described in FIGS. 2A and 2B with 30 or 300 μg PLA2 or ovalbumin, respectively, six weeks after the last sensitization injection. After 30 minutes, we analyzed anaphylaxis as changes in rectal temperature. Mice that had been sensitized with native BV (FIG. 2C) or ovalbumin (FIG. 2D) had a profound drop in body temperature. In contrast, mice that were originally sensitized with the heat-denatured formulation had developed less allergy and consequently showed smaller changes in body temperature (p=0.035 for BV and p=0.050 for ovalbumin).

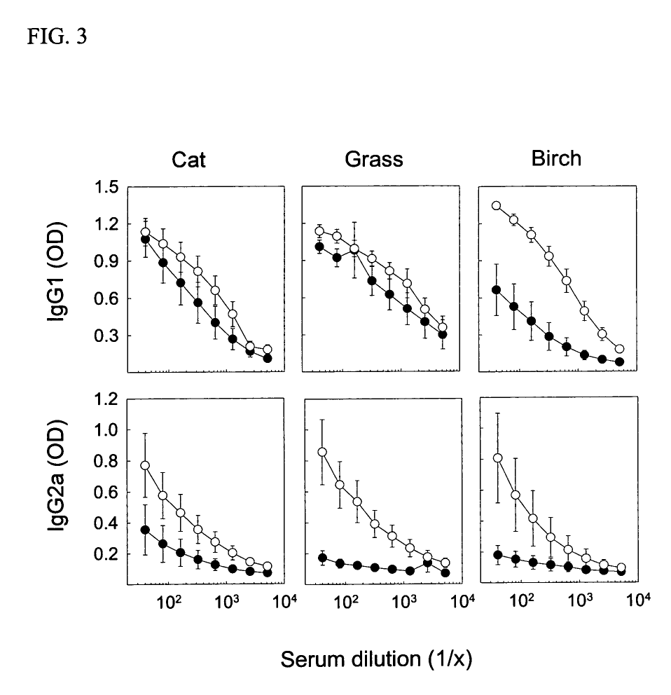

The effect of heat-denaturation was further demonstrated with other allergens, such as cat dander, grass pollen, and birch pollen allergen extracts (FIG. 3). After heat-denaturation, also these allergens also caused highly increased IgG2a production in mice that received the allergens by intralymphatic injections. With the exception of birch pollen, which showed increased IgG1 immunogenicity after denaturation, the other allergens gained no IgG1 benefit by heat-denaturation.

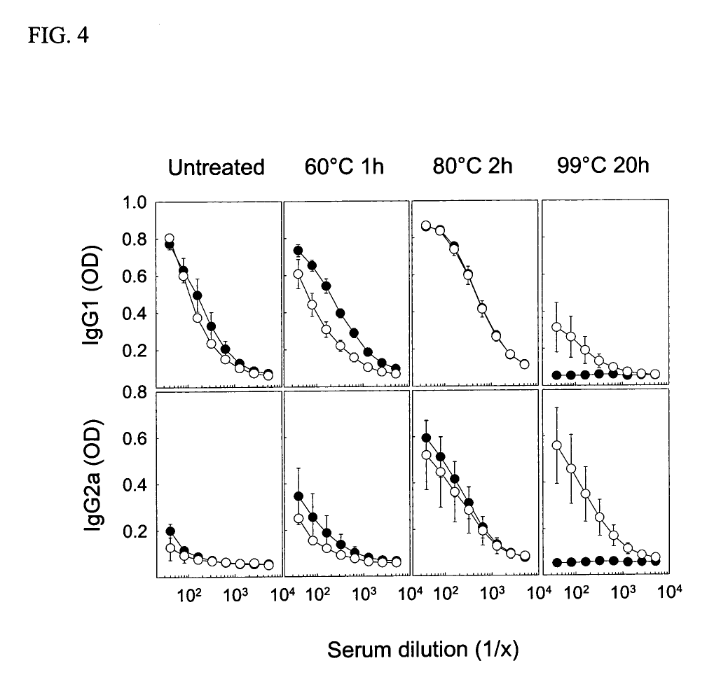

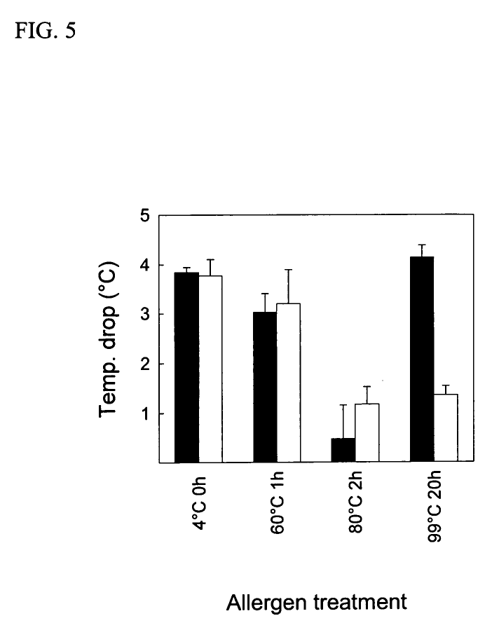

To test whether an optimum condition for heat-denaturation existed or whether the IgG2a responses continuously increased as a function of temperature and time, we immunized mice intralymphatically as described above with 0.3 μg untreated BV or BV heated at 60, 80 or 99° C. for 1, 2, and 20 hours, respectively. The results again showed that heating at 60° C. and 80° C. increased the BV immunogenicity for IgG2a, but not for IgG1. However, treatment at 99° C. and for 20 h fully abolished BV immunogenicity. This can be partly (IgG1) or fully (IgG2a) restored by the addition of a minute amount of native BV (0.03 μg). Whereas immunization with the fully destroyed BV caused anaphylaxis in PLA2-challenged mice, the addition of a minute amount of native BV prevented anaphylaxis (FIG. 5). The addition of 0.03 μg native BV to 0.3 μg untreated (4° C.) or partly treated (69° C. and 80° C.) BV did not further improve protection (FIGS. 4 and 5).

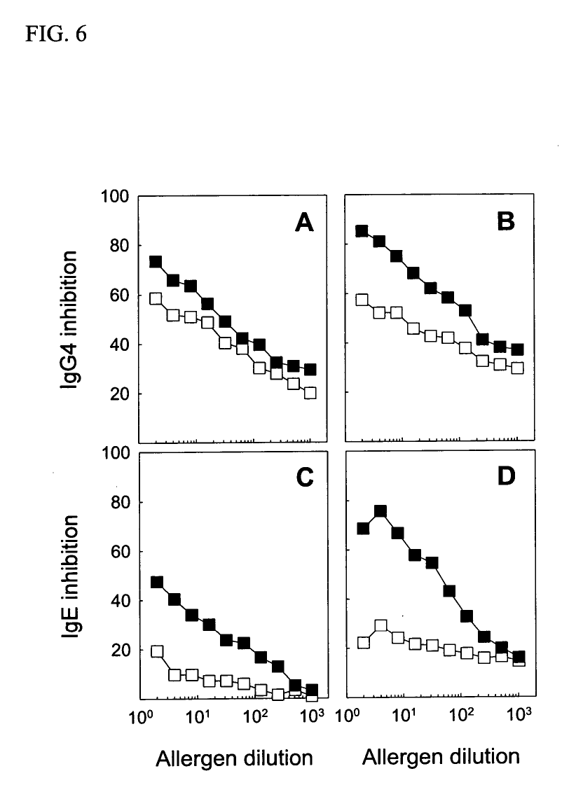

The human immunoglobulin recognition of native and denatured PLA2 or bee venom was analyzed by competitive inhibition assay using human antibodies specific for IgG4 and IgE. Both native and denatured allergens are recognized by human IgG4 (FIG. 6A-B), results which were verified by Western blot analysis. However, heat-denaturation of the allergens virtually abolished their IgE-binding capacity (FIG. 6C-D).

1. A composition for desensitizing an individual to an allergen, comprising:
a heat-denatured allergen composition comprising an allergen selected from the group consisting of bee venom, grass pollen, birch pollen, and cat dander or hair; and
a physiologically acceptable vehicle.

2. The composition of claim 1 wherein the heat-denatured allergen composition comprises an extract of bee venom.

3. The composition of claim 1 wherein the heat-denatured allergen composition comprises an extract of birch pollen extract.

4. The composition of claim 1 wherein the heat-denatured allergen composition comprises an extract of grass pollen.

5. The composition of claim 1 wherein the heat-denatured allergen composition comprises an extract of cat dander or hair.

6. The composition of claim 1 wherein the heat-denatured allergen composition comprises a purified allergen selected from the group consisting of phospholipase A2, hyaluronidase pi m 6, Fel d 1, cystatin, Phl p 1, Phl p 2, Phl p 4, Phl p 5, Phl p 6, Phl p 7, Phl p 11, Phl p 12), Bet v 1, and Bet v 2.

7. The composition of claim 1 which is pyrogen-free.

8. The composition of claim 1 further comprising an adjuvant.

9. A method of preparing composition for desensitizing an individual to an allergen, comprising:
heating an allergen preparation comprising an allergen selected from the group consisting of bee venom, grass pollen, birch pollen, and cat dander or cat hair for a time sufficient to (a) reduce the allergen's ability to bind IgE, (b) reduce the allergen's ability to induce production of IgE, and (c) increase the allergen's ability to induce production of an IgG, thereby forming a heat-denatured allergen composition.

10. The method of claim 9 wherein the allergen preparation comprises a purified allergen selected from the group consisting of phospholipase A2, hyaluronidase pi m 6, Fel d 1, cystatin, Phl p 1, Phl p 2, Phl p 4, Phl p 5, Phl p 6, Phl p 7, Phl p 11, Phl p 12), Bet v 1, and Bet v 2.

11. The method of claim 9 further comprising combining the heat-denatured allergen composition with a physiologically acceptable carrier.

12. The method of claim 9 wherein the allergen preparation is heated at a temperature between 60 and 99 °C.

13. The method of claim 9 wherein the extract is heated at a temperature between 40 and 99° C.

14. The method of claim 9 wherein the extract is heated at a temperature between 40 and 121° C. or 180° C.

15. A composition for desensitizing an individual to an allergen made by the method of claim 9.

16. A method of desensitizing an individual to an allergen, comprising:
administering to the individual an effective amount of the composition of claim 1.

17. The method of claim 16 wherein the composition is administered intranodally.

18. The method of claim 16 wherein the individual is a human.

19. A method of desensitizing an individual to an allergen, comprising:
administering to the individual an effective amount of the composition of claim 15.

20. The method of claim 19 wherein the composition is administered intranodally.

21. The method of claim 19 wherein the individual is a human.
